(19) United States
(12) Patent Application Publication
(10) Pub. No.: US 2011/0044958 A1
(43) Pub. Date: Feb. 24, 2011

(54) ACTIVATED MESENCHYMAL STEM CELLS FOR THE PREVENTION AND REPAIR OF INFLAMMATORY STATES

(75) Inventors: Amelia Bartholomewq, Lake Forest, IL (US); David Polchert, Park Ridge, IL (US); Erzsebet Szilagyi, Chicago, IL (US)

Correspondence Address: BARNES & THORNBURG LLP, P.O. Box 2786, CHICAGO, IL, 60690-2786, US

(73) Assignee: The Board of Trustees of the University of ILLINOIS

(21) Appl. No.: 12/922,417

(22) PCT Filed: Mar. 16, 2009
(86) PCT No.: PCT/US09/37299

§ 371 Date: Nov. 9, 2010

Related U.S. Application Data
(60) Provisional application No. 61/036,808, filed on Mar. 14, 2008.
(60) Provisional application No. 61/049,249, filed on Apr. 30, 2008.

(51) Int. Cl. A61K 35/32; A61K 35/12; C12N 5/0775

(52) U.S. Cl. 424/93.7; 435/375; 435/372.1

(57) ABSTRACT

Inflammatory cytokines e.g. IFN-γ serve as initiating stimuli for MSC immunosuppressive activity in vivo. Other inflammatory cytokines, such as TNF alpha, the molecule hemoxygenase I, and TLR ligation of MSC may also provide such a response.

This invention was made with U.S. government support under Grant No. H8810 awarded by IRMI and Grant No. E6614 awarded by NIH. The U.S. government has certain rights in the invention.

### BACKGROUND

Inflammatory cytokines e.g. IFN-γ serve as initiating stimuli for MSC immunosuppressive activity in vivo. Other inflammatory cytokines, such as TNF alpha, the molecule hemoxygenase I, and TLR ligation of MSC may also provide such a response.

Graft versus host disease, mediated by donor T cells, is a significant source of morbidity and mortality following allogeneic stem cell transplantation. Mesenchymal stem cells (MSC) can successfully treat ongoing graft versus host disease, presumably due to their ability to suppress donor T cell proliferation. However, little is known about the mechanisms which MSC exert in vivo to prevent graft versus host disease.

Allogeneic hematopoietic stem cell transplants have the potential to play a significant curative role in the treatment of malignant and non-malignant hematopoietic disorders, autoimmune diseases, immunological deficiencies, and in the induction of transplantation tolerance. Unfortunately, widespread application of this therapeutic modality is limited due to the morbidity and mortality of graft versus host disease (GVHD), which affects 50% of stem cell transplant recipients. While grafts highly matched to the recipient, young donors, donor/recipient sex match, and post-transplant immunosuppression are strategies used to reduce the risk of GVHD, thus far, the greatest preventative measure has been intentional underutilization of stem cell transplantation. Theoretically, strategies aimed in preventing GVHD would target early initiating factors either during the inflammatory milieu created in the wake of tissue damage from conditioning regimens, or during T cell antigen recognition and proliferation. After the efferent effector phase occurs, donor T cell mediated destruction of host tissues occurs and preventive strategies are replaced with treatment regimens.

Mesenchymal stem cells (MSC) have been used in the efferent phase of GVHD to treat ongoing, acute, steroid resistant GVHD. In contrast, when given at the time of bone marrow transplant, for the prevention of GVHD, the incidence of grade III/IV GVHD was not significantly improved. MSC reliably suppressed large scale T cell proliferation in response to polyclonal stimulation in vitro. In contrast, with allogeneic mixed lymphocyte cultures of variable stimulation, MSC suppression is also variable; MSC do not completely abrogate lymphocyte proliferative responses between all donor and recipient pairs. In addition, MSC do not suppress the modest T cell proliferative response to recall antigens.

Murine experimental models used to dissect the mechanism of MSC effects in the course of GVHD have yielded mixed results, with some studies showing MSC efficacy and others finding no effect. Several factors are likely to contribute to the variable results. MSC tissue source, (ie, bone marrow, cord blood, adipose tissue), method of isolation to remove myeloid precursors (several weeks vs rapid immunodepletion) and timing of MSC administration are potential variables which could explain these differences. Notably, such variation has not been observed clinically, with MSC treatment of ongoing GVHD reported to have significant efficacy, while MSC prevention of GVHD not shown to be effective clinically.

### SUMMARY

Use of activated mammalian mesenchymal stem cells to prevent graft versus host disease in a mammal as result of a transplant is disclosed. The steps include:

(a) activating the mammalian mesenchymal stem cells with interferon gamma (IFN-γ); and

(b) administering the activated mammalian stem cells to the mammal who will host the graft. Activating was achieved by a high dose of interferon gamma/ml, wherein a high dose is greater than 50 units interferon gamma/ml. A preferred dose is about 2,000 or 5,000 units administered for 2 hours, or 500 units administered for 6 days.

The mesenchymal stem cells were from bone marrow, e.g., human bone marrow, but other sources are suitable such as placental or adipose tissue are suitable.

Administering may be approximately at the time of, or after, the transplant.

The composition is useful for the preparation of a pharmaceutical composition for preventing graft versus host disease in a mammal as result of a transplant. A composition that improves the outcome of organ transplantation in a host, includes mammalian, e.g., human mesenchymal stem cells, activated by an inflammatory cytokine, e.g., IFN-γ. The composition is also useful to initiate angiogenesis and may be administered to a subject in need of tissue regeneration or organ repair.

A method to produce activated mesenchymal stem cells for use in tissue transplantation includes:

(a) obtaining mesenchymal stem cells; and

(b) activating the mesenchymal stem cells by culturing the cells with IFN-γ.

Activating is achieved with a dose greater than 50 units interferon gamma/ml given at least 2 successive pulses of exposure to >50 units/ml IFN-γ within a 3 day-7 day period.

The mesenchymal stem cells are from bone marrow, e.g., human bone marrow, and other suitable sources.

### BRIEF DESCRIPTION OF THE DRAWINGS

FIG. 1. MSC phenotype following rapid immunodepletion: when compared to isotype controls, MSC stained positive for mhc class i (h-2k^{d}), the vla complex marker cd29, the cell adhesion receptor cd44, and the hematopoietic stem cell marker sca-1. mscs stained negative for mhc class ii (i-a^{d}), macrophage cell surface markers (cd11b, cd14), b cell marker (b220), lymphocytes (cd2, cd3, cd4, and cd8a), and the hematopoietic stem cell markers thy-1 and c-kit. histograms represent consistent findings after greater than 30 isolation experiments.

FIG. 2. The effect of timing of MSC administration on 40-day survival following induction of GVHD. Following lethal irradiation, B6 recipients underwent transplantation with allogeneic Balb BMC and splenocytes on day 0 (control, solid line) resulting in 30% survival, (experiment repeated 6 times, n=20). In experimental groups (dashed line), Balb MSC were administered on days 0 (2A), 2 (2B, n=20), 20 (2C) or 30 (2D). MSC administered on days 2 and 20 significantly increased survival from 10% to 60% and 10% to 50%, respectively (p<0.05).

FIG. 3. Effect of MSC dose escalation. Low (0.1×10⁶) and high (0.5×10⁶) dose MSC were administered on day 2 (3A) or day 20 (3B). Both low and high doses led to a significant improved survival when given on either days 2 or 20. High dose MSC given on day 20 significantly increased survival to 85% (p=0.006), when compared to control animals.

FIG. 4. GVHD scores of lung, colon, spleen, and skin. Recipients of 0.5×10⁶ or 0.1×10⁶ MSC and control recipients were euthanitized for histological examination. Sections taken from lung, colon, and spleen were scored on a scale of 1 to 4 with 4 being the most severe changes consisting with tissue destruction observed in GVHD. Skin was scored on a scale of 1 to 3, with 3 being the most severe. This experiment was repeated three times (n=3 per group).

FIG. 5. Requirement of IFN-γ for MSC mitigation of GVHD. Splenocytes from IFN-γ knock out mice were used to induce GVHD (solid line). Addition of MSC on Day 2 failed to affect survival (dashed line), indicating MSC required IFN-γ to initiate their suppressive effects.

FIG. 6. Activation of MSC with IFN-γ. Either untreated (6A, dashed grey line) or IFN-γ treated Balb MSC were administered on day 0. MSC treated with 5 (solid grey line) or 50 units (dashed black) IFN-γ showed no effect on GVHD-related mortality when compared to untreated MSC. MSC treated with 500 units (solid black line) were significantly more effective than untreated MSC (p=0.006) and MSC treated with lower doses of IFN-γ. IFN-γ treatment appeared to have a direct effect on MSC, (6B), causing new expression of MHC Class II, changing both the phenotype and function of these cells. No detection of CD45 or CD11b populations were noted after IFN-γ treatment (grey line) when compared to pre-treatment (black line), indicating that IFN-γ treatment did not expand an immunoregulatory dendritic cell population, (experiment performed >10 times prior to each transplant). Following transplantation and the development of GVHD, circulating IFN-γ measured in the serum by ELISA (6C) surpassed 500 units by day 7 with a gradual drop below 500 by days 21 and 30 suggesting there was sufficient circulating IFN-γ to activate MSC by day 7, but not after day 30, (each timepoint represents analysis of 3-5 recipients measured in duplicate).

FIG. 7. MSC exposed to 6 or 7 days of high dose IFN-γ show significantly increased levels of the immunosuppressive cytokine, TGF-beta, and pro-angiogenic factors VEGF and HOF.

FIG. 8. (A) MSC stained for fibronectin (arrow) and DAPI (nuclei), (B & D) MSC-islet co-culture showing a single islet stained for insulin (arrow), fibronectin (arrow), DAPI (nuclei), (C) MSC-islet co-culture stained for insulin (arrow), and nuclear stain for MSC (arrow) after two day co-culture. MSC show a circular pattern of congregation around islets (C & D).

FIG. 9.A is a staining of CD31+ cells (PE-red, white arrows show clusters of CD31+ staining cells in heart endothelium) in allogenic graft and B) autologous interferon gamma activated MSC treated recipient's graft 7 days after transplantation (40×). (IIFC-FR with anti CD 31 rat monoclonal antibody PE conjugated 1:50 dilution, Abcam);

FIG. 9B is a quantitative analysis of CD31+ signal in transplanted heart. Fluorescence signal positive objects were counted in 5 standar ROI in 3 sections of each graft (n+2) in the following MSC treatment, donor MSC treatment, no MSC treatment and naïve non transplanted graft. MSC exposed to 6 or 7 days of high dose IFN-gamma show significantly higher numbers of endothelial cells within the transplanted murine heart allograft when compared to no treatment or non-activated MSC. When compared to non-treated or non-activated MSC treated (FIG. 9A, left panel), recipients of activated MSC show increased CD31 staining in allogeneic transplanted hearts when studied 7 days after transplant (shown in arrows, right panel, FIG. 9A). The number of CD31+ cells were measured using confocal microscopy and compared to naïve hearts which were never transplanted, hearts treated with no MSC, and hearts treated with either activated donor or host derived MSC (FIG. 9B). There was a statistically increased number of CD31+ cells when compared to control groups. In addition, when comparing the number of endothelial cells in MSC treated grafts to naive hearts, there was and no significant difference between these two groups, suggesting that MSC-induced repair and regeneration was promoting an allograft condition which most resembled a naïve, non-transplanted heart.

FIGS. 10 A & B MSC exposed to 6 or 7 days of high dose IFN gamma induce tolerance to allogeneic heart transplants, whereas non-activated MSC do not. B6 MSC were administered to B6 recipients of Balb hearts. Donor MSC and non-activated MSC prolonged survival for up to 40 days, however no tolerance was observed (FIG. 10A). When rapamycin was added for a 5 day course post-transplant, this treatment alone did not lead to tolerance. When rapamycin was combined with activated MSC, greater than 100 day survival was achieved leading to tolerance of the heart allografts (FIG. 10B)

FIG. 11 MSC exposed to 6 or 7 days of high dose IFN gamma show significant changes in functioning production of chemokines, cytokines and growth factors when compared to MSC exposed to lower (50 units/ml) or no IFN gamma. Specifically, in the first 24 hours following tissue injury as observed in wounds or following organ transplant, chemokines which recruit neutrophils and macrophages predominate. Interferon gamma activated MSC with either 50 units interferon (square), 500 units interferon (triangle) or no interferon gamma (diamond) demonstrate distinct differences in production of chemokines which recruit these cells, such as KC, LIX, and MIP-2, all of which are elevated at day 6 of incubation with interferon but show no baseline activity with non-activated MSC. In the next 3-7 days following tissue injury, lymphocytes are recruited to the site of injury with chemokines such as IP-10, MIG, MIP-1b. Once again there is a distinct difference in expression of these cytokines between MSC transformed with higher doses of interferon gamma than those MSC with no interferon gamma These differences punctuate a functional difference in MSC exposed to high dose interferon gamma when compared to non-exposed MSC.

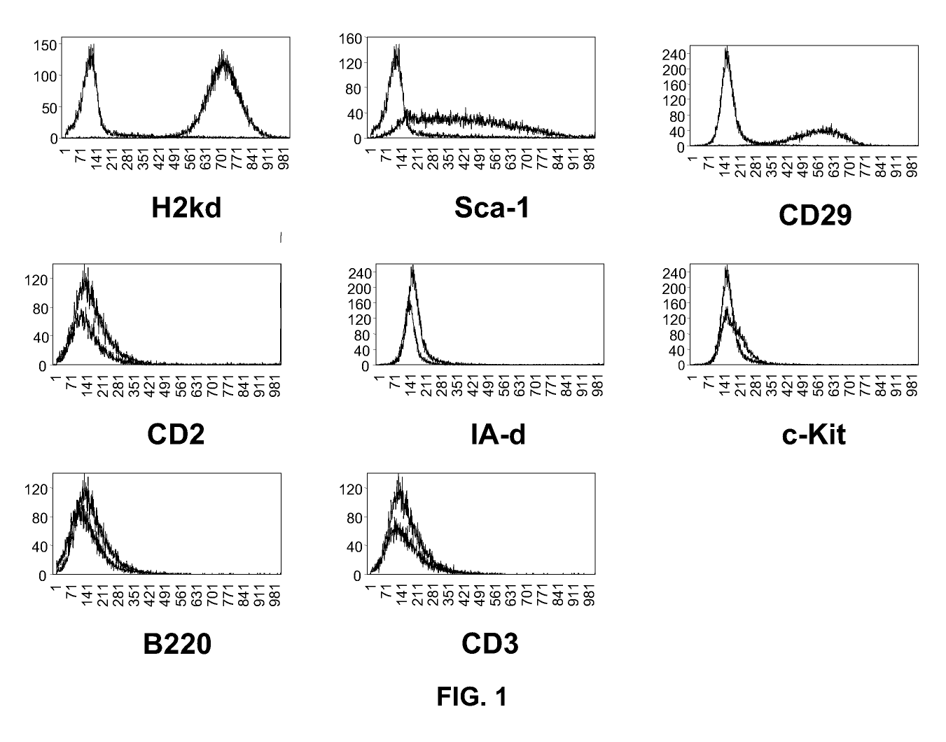

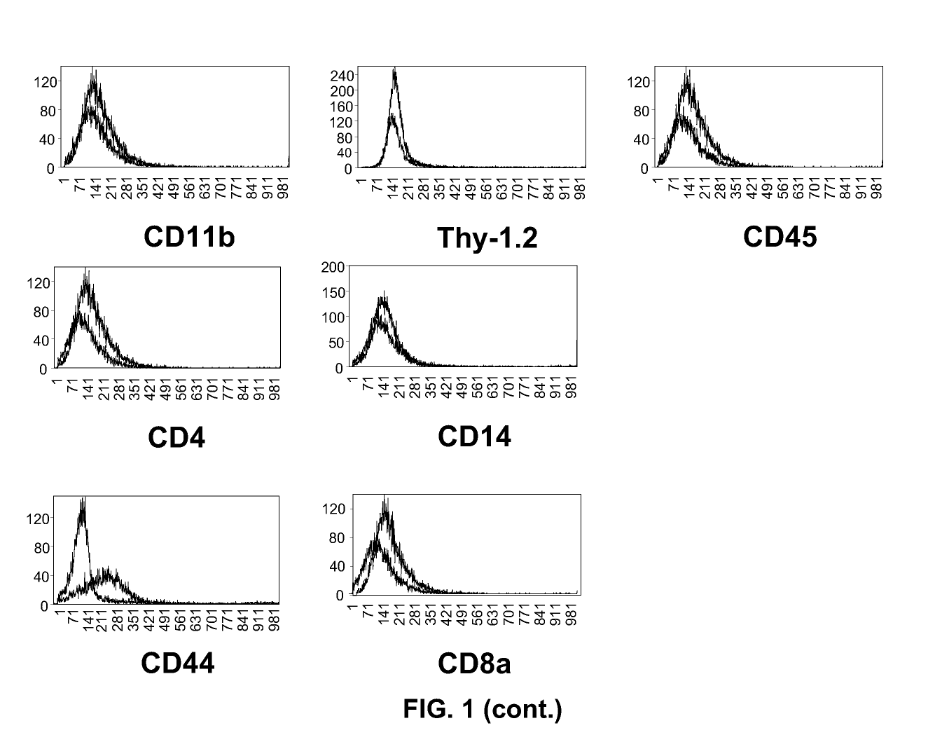

### DETAILED DESCRIPTION

Surprisingly, inflammatory cytokines serve as initiating stimuli for mesenchymal stem cell (MSC) immunosuppressive activity in vivo. Bone marrow isolated MSC activated by IFN-γ suppressed the development of graft versus host disease (GVHD) when given at the time of the bone marrow transplant as a preventive measure. Further, if given at later timepoints, it is 5 times more efficacious than non-activated MSC. IFN-γ activation was required to initiate MSC efficacy with a phenotypic change of MSC to express MHC Class II molecules remaining lineage negative. Cells remained devoid of expression of T, B, or macrophage cell surface markers, as well as the hematopoietic marker, CD45. IFN-γ induced MSC activation led to Class II expression on the surface of MSC.

MSC, pre-treated with IFN-γ, suppressed GVHD more efficiently than 5 fold greater numbers of MSC that were not activated. When given at the time of bone marrow transplantation, activated MSC prevented GVHD mortality (100% survival, p=0.006). MSC activation was dependent on the magnitude of IFN-γ exposure, with increased IFN-γ exposure leading to increased MSC suppression of GVHD. Activated MSC are a new strategy for preventing GVHD requiring smaller numbers of MSC.

Increased immunosuppressive properties of activated MHC Class II+ MSC can be useful in organ transplantation for the induction of tolerance or reduction of ischemia reperfusion injury, mitigation of autoimmune disorders.

Activated MSC provide increased deposition of matrix as well as pro-angiogenic factors. These qualities are likely to facilitate tissue regeneration as well as organ repair.

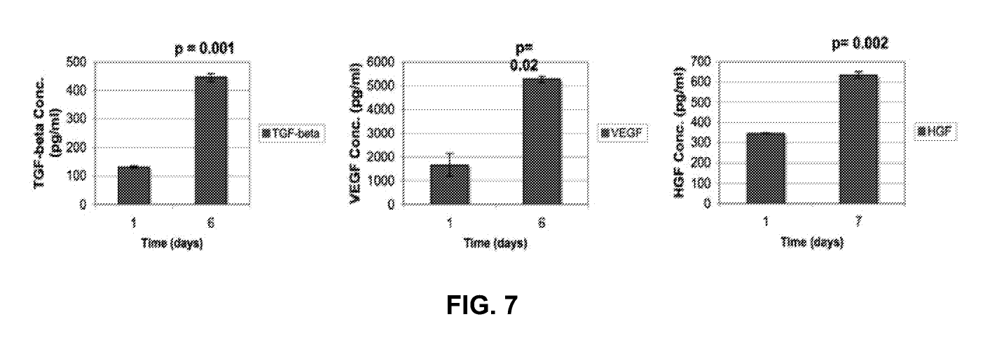

In studies with activated MSC, activation led to greater deposition of matrix. An example of how increased matrix might affect tissue regeneration is given in the studies undertaken with islets and MSC. Islet production of insulin is dependent on matrix. Therefore to test whether such increases in matrix by activated MSC could improve islet function, Murine Balb/c donor islets were isolated and co-cultured with C57/B6 MSC (FIG. 7). To test whether MSC co-culture resulted in increased matrix deposition, MSC-Islet co-culture (FIGS. 7B & D) was stained for fibronectin and compared by digital quantification to MSC (FIG. 7A) or islets alone. There was increased deposition of fibronectin when compared to non-activated MSC or islets alone, with fibronectin observed to congregate around the islet. MSC changed their wide distribution, migrating and binding to the islets (as evidenced by the lack of MSC in between the islets and the greater concentration of MSC around the islets) (FIGS. 7C & D). Islets co-cultured with MSC significantly increased insulin production when compared to islets alone in vitro. These data indicate that MSC can provide matrix structural support for transplanted tissues and the presence of this support aids in more efficient tissue function and regeneration.

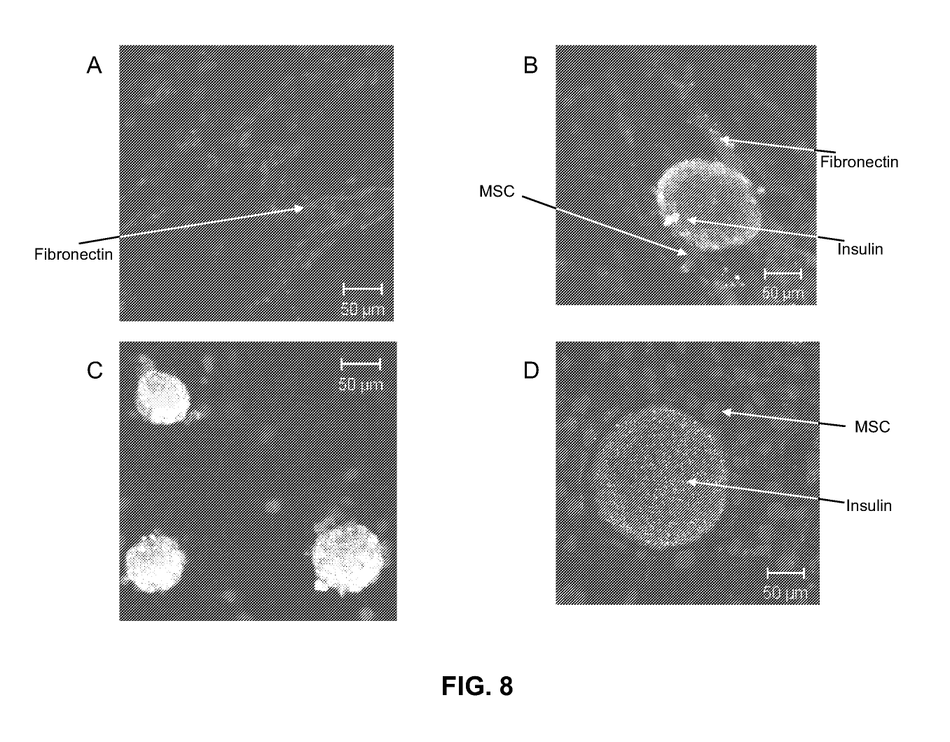

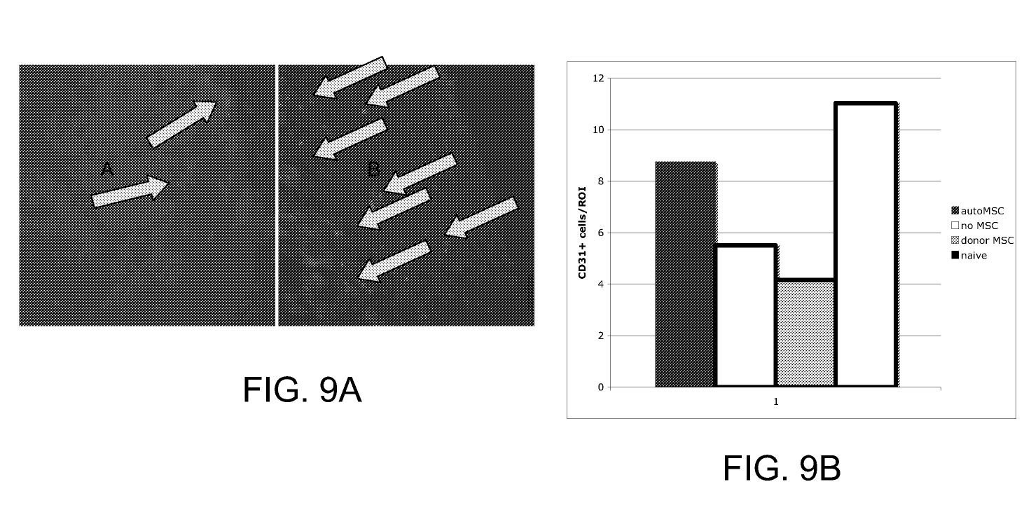

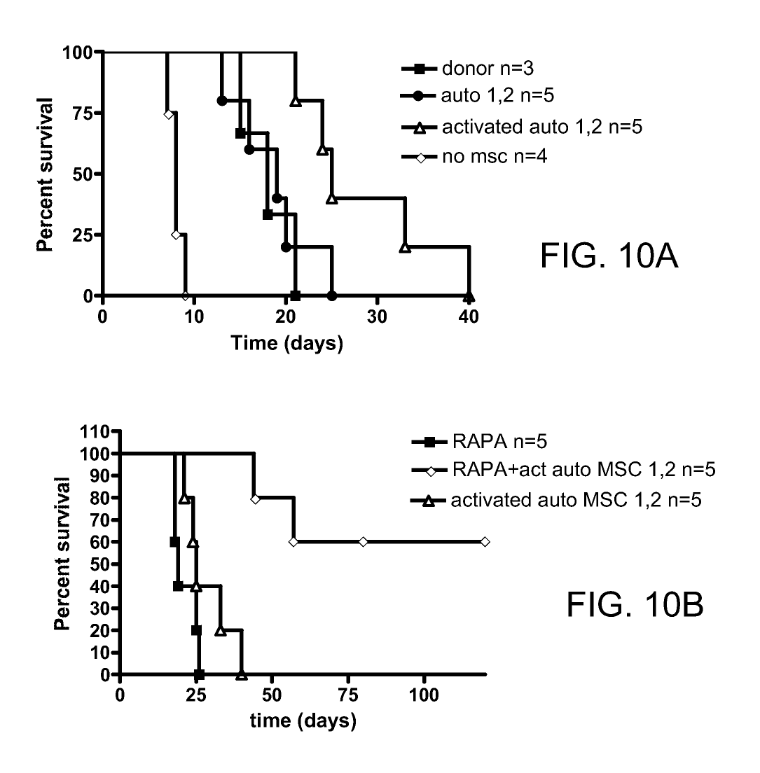

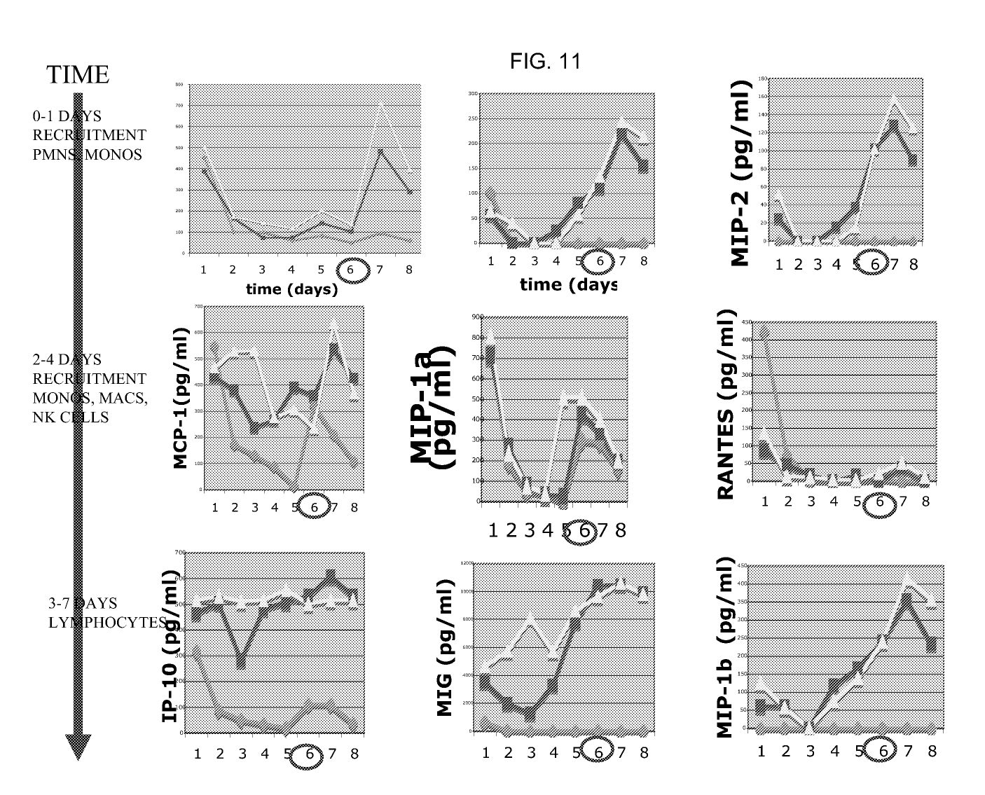

TGF-beta, VEGF, and HGF all significantly increase in production (FIG. 8) following activation with 6 or 7-day culture with IFN-γ. Using the 6 day co-culture with interferon gamma, these cells change in substance to express MHC Class II and to become more 1) immunosuppressive promoting GVHD prevention in vivo 2) produce greater amounts of chemokines attracting neutrophils, macrophages and then subsequently efficiently providing suppression of these cells within an allograft transplant, 3) induce high numbers of CD4 T-regulatory cells immediately after infusion, 4) express high amounts of pro-angiogenic factors such as VEGF, HGF.

A rapid immunodepletion method to isolate murine MSC resulted in dramatically and unexpected shortened culture times and low passage cells. Efficacy of the MSCs for GVHD treatment were similar to clinical observations, with lack of efficacy in prevention and improved survival when given during ongoing GVHD. To further dissect the factors which might initiate MSC suppression of GVHD, their earliest timepoint of efficacy was localized to occur following antigen presentation. Because IFN-γ is produced by donor T cells in response to antigen recognition, it could initiate MSC efficacy in vivo. A dose response effect was observed, with higher doses of IFN-γ being more effective than lower doses. IFN-γ serves as an initiating stimulus for MSC immunosuppressive activity in vivo. MSC response to this pro-inflammatory cytokine is differential, with three-log increases in IFN-γ required for maximal T cell suppression in vivo. MSC exposure to concentrated amounts of IFN-γ can stimulate MSC to prevent GVHD and provides the basis for a new strategy in prevention of GVHD.

Following analysis of recipient sera, sufficient serum levels were present to stimulate MSC on day 7. This suggests, that in the mouse model, this time period is likely to be effective for MSC activation. Correlation with IFN-γ serum levels in patients may guide the timing of MSC therapy. A lack of stimulating levels of IFN-γ was observed on days zero, one, or two. Threshold levels were present in areas where newly activated dendritic and T cells were producing high levels of IFN-γ locally, such as the spleen and lymph nodes. It has been reported that following administration, T cells migrate to secondary lymphoid organs and target organs which predominantly express certain chemokines and chemokine receptors, such as MIP-1 alpha, MIP-2, MCP-1, and MCP-3. MSC migration can also be enhanced by these chemokines and MSC chemokine receptor expression can be regulated by interferon gamma. Taken together, it is possible that, following infusion, MSC migrated in response to tissue chemokine expression, where local production of IFN-gamma by activated dendritic cells and donor T cells, during early GVHD (day 2) was sufficiently concentrated to provide MSC activation. Following activation, MSC have the capability to inhibit both dendritic cell and early T cell responses. It is possible that part of the efficacy of day 2 administration is due to the ability of MSC to dampen the escalation of GVHD by local control within both the target organs as well as the lymphoid organs. Of all the tissues examined, splenic tissue appeared to have some of the most significant improvement following MSC treatment. The powerful and seemingly preferential effect of MSC observed on this lymphoid tissue, may reflect the greater levels of locally produced IFN-γ during GVHD.

MSC administered on day 30 had no efficacy when compared to day two or day 20. This observation may be due to two factors: the overwhelming increase in the number of donor T cells for which the number of MSC were insufficient and/or the corresponding drop in the levels of IFN-γ. Despite such increased numbers of T cells, T cell production of IFN-γ has been observed to decrease during ongoing GVHD. In addition, low serum levels of IFN-γ were observed. It is possible that MSC administered on day 30 failed to receive sufficient IFN-γ, either through the circulation or through local production. The lack of available IFN-γ to MSC may have limited their ability to produce significant amounts of immunosuppressive molecules such as IDO, IL-10, TGF-β. All of these have been observed to have a dose response relationship with IFN-γ treatment of MSC. In vivo observations are very similar to those observed in vitro; using this model, MSC-induced suppression of GVHD is dependent on the magnitude of IFN-γ stimulus.

Addition of five-fold greater MSC, 0.5×10⁶ MSC, improved survival to 85% when MSC were administered on day 20. When MSC were administered on day 2, survival remained at 60% despite the higher dose. MSC given on day 2 failed to receive sufficient amounts of IFN-γ to become activated. Following activation of MSC, 0.1×10⁶ MSC administered on day 2 improved survival to 100%. This observation suggests that the efficacy of MSC can be manipulated by IFN-γ activation. Since IFN-γ production can vary during the course of GVHD, the efficacy of MSC may also vary unreliably, with some treatments not attaining full immunosuppressive potential. Depending on the number of activated T cells, the ratio of T cells to MSC, and the available IFN-γ, it is possible that lack of optimization of these three factors could result in MSC therapy which is only marginally beneficial.

With the incidence of GVHD exceeding 50%, and success in GVHD prevention being limited, new strategies in preventing GVHD and its significant morbidity and mortality are needed. Prevention of GVHD with activated MSC may play a role in broadening the therapeutic potential of allogeneic stem cell transplantation.

One of the strategies for control and/or prevention of GVHD has been to induce a shift from TH1 to TH2 cytokines. Because IFN-γ is a known stimulant of TH1 cytokine production, a potential harmful side effect from IFN-γ MSC treatment might be the release TH1 cytokines, IL-2, GM-CSF, and TNF-α. Analysis of day 1 and day 6 supernatants from MSC exposed or not exposed to 500 units/ml of IFN-γ (Biosource, 20-plex cytokine detection kit, Invitrogen) showed undetectable amounts of TNF-α and GM-CSF and only modest increases in IL-2 from 50 pg/ml to 80 pg/ml in IFN-γ MSC. Untreated MSC had undetectable amounts of all three TH1 cytokines. IFN-γ had no effect on inducing IL-10 production, but significantly increased TGF-beta (p=0.001). TGF-beta has been implicated in T cell suppression by MSC. In response to IFN-γ, MSC may increase suppression and limit TH1 responses.

### Example 1

### Isolation of MSC by Rapid Immunodepletion

Murine MSC were isolated using rapid immunodepletion of macrophages. This technique also avoided long-term exposure of primitive MSC to mature myeloid lineages, which may enable murine MSC to become more immunostimulatory than immunosuppressive in vivo. These cells were observed to be capable of differentiating into adipogenic and osteogenic tissues.

### Example 2

### Treatment of GVHD

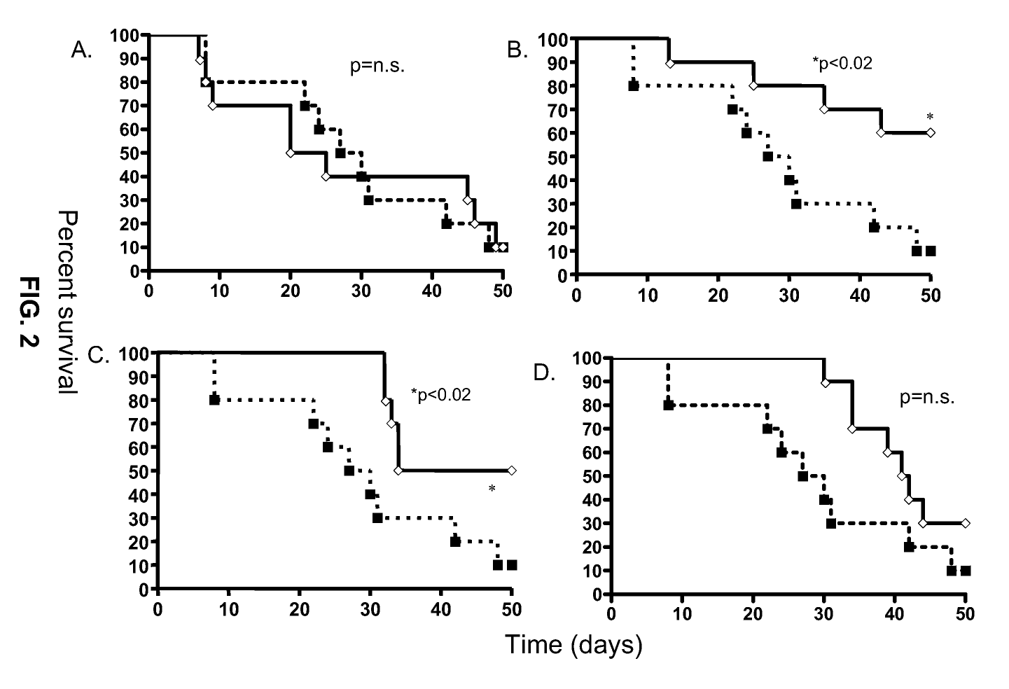

To establish whether MSC isolated, according to the methods disclosed herein proved efficacious in the treatment of GVHD, 0.10×10⁶ MSC were administered during various phases of GVHD. First, MSC were administered on day 0 along with the bone marrow graft and supplementary T cells to induce GVHD (FIG. 2A). For this administration, MSC were co-cultured with the bone marrow graft and splenocytes for two hours prior to administration. Cell contact between MSC and GVHD-producing T cells prior to donor antigen recognition suppressed T cell activity and subsequent GVHD mortality. Pre-emptive cell contact of MSC with T cells did not prove to be effective; there was no statistical improvement in GVHD-related mortality when compared to control animals that received bone marrow grafts and supplementary T cells.

### Example 3

### Treatment of GVHD

During Phase II of GVHD, donor T cells are exposed to host antigens and become active, serving to both proliferate and recruit additional T cells. MSC, administered after donor antigen recognition, could mitigate GVHD mortality (FIG. 2B). Antigen recognition and/or subsequent activation of T cells appeared to be required for MSC efficacy since MSC given on day 2 increased survival from 10% to 60%, (p<0.02). Following MSC infusion, some of the animals that had developed signs of GVHD, such as ruffled fur and alopecia, had improvement of these physical findings. Many surviving animals experienced a complete reversal to normal appearing fur.

### Example 4

### Treatment of Ongoing GVHD

MSC were tested for their ability to treat ongoing GVHD (administered on day 20) or treatment of severe, pre-morbid GVHD (given on day 30). MSC administration increased survival from 10% to 50% when given on day 20 (p<0.02, FIG. 2C), and to 20% for day 30 treated animals (p=0.08, n.s, FIG. 2D). These data show MSC isolated with rapid immunodepletion are effective in preventing GVHD as well as treating ongoing GVHD. MSC contaminated with >3% CD45+ cells and MSCs of late passage (greater than 6), had no significant effect on GVHD-related mortality, indicating that early passage and significant immunodepletion were required for MSC suppression of GVHD.

### Example 5

### MSC Treatment is Dose-Dependent

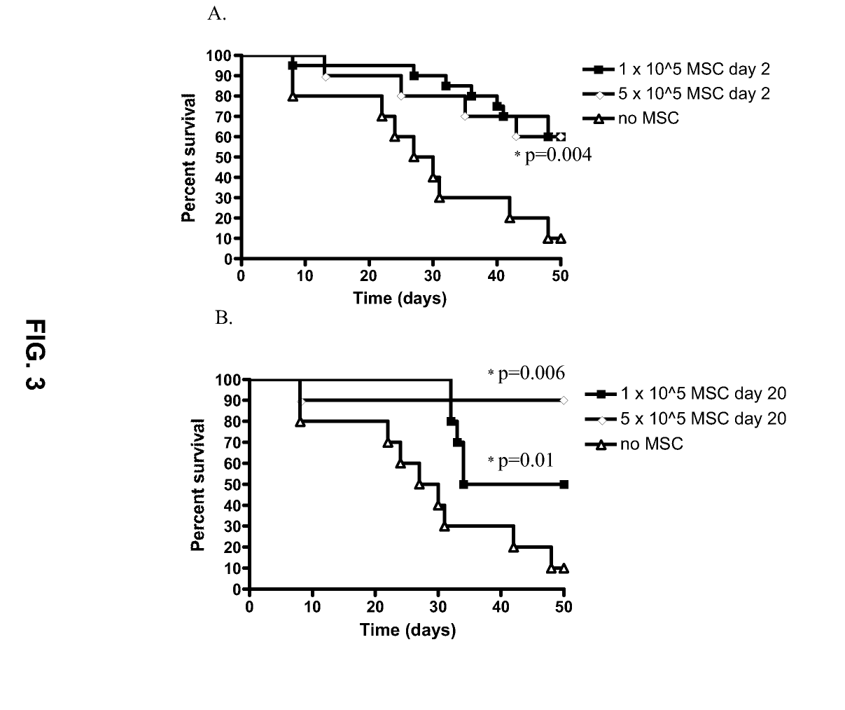

To further define the limits of MSC efficacy, the effect of MSC dose on survival was examined. Following transplantation, either 0.1×10⁶ or a five-fold greater dose, 0.5×10⁶ MSC were administered on either day 2 or day 20 (FIG. 3) and compared to transplanted animals which did not receive MSC. There was no dose-response effect when a higher dose was administered on day 2. While both 0.1×10⁶ and 0.5×10⁶ MSC significantly increased survival (p=0.004), the two survival curves were indistinguishable from each other (FIG. 3A). These data indicated that higher doses, when given as a preventative measure, did not appear to change the course of mortality. For animals receiving MSC on day 20, survival following 0.5×10⁶ MSC significantly increased from 10% to 85%, (p=0.0006, FIG. 3B).

Statistical comparison between low and high dose revealed a strong trend suggesting a difference between the two groups, (p=0.07). Based on these data, it appeared that MSC behaved differently when given for treatment than when given as a preventative measure. Because higher numbers of T cells are likely to have undergone antigen recognition and proliferation on day 20 when compared to day 2, the difference in MSC behavior was likely due to an increase in the magnitude of activating signals generated from the increased antigen presentation and/or T cell proliferative activity.

### Example 6

### Histological Findings

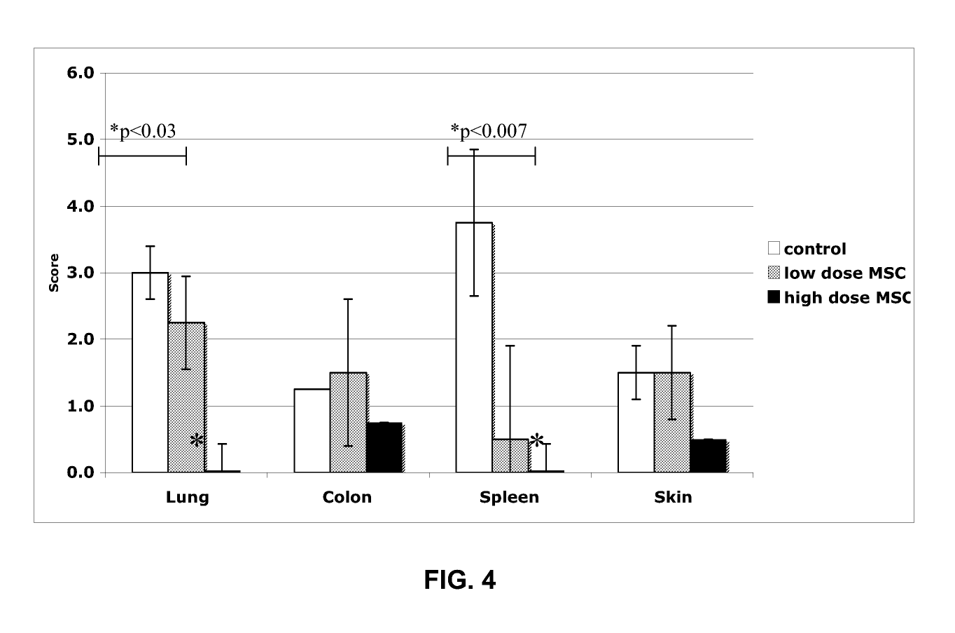

Tissues were analyzed from recipients who underwent treatment with MSC. On histological examination of lung, spleen, colon, and skin, MSC treatment improved the severity of GVHD scoring. Spleen and lung displayed the greatest findings, with both tissues observed to be normal appearing after receiving 0.5×10⁶ MSC (p<0.0007, and 0.03, respectively, FIG. 4).

### Example 7

### IFN-γ is Required for MSC Reduction of GVHD Mortality

To test whether antigen recognition and potentially, T cell proliferation was required to initiate MSC activity, the regulatory protein Interferon gamma (IFN-γ) was tested for its ability to initiate suppressive activity in MSC. This protein was chosen due to several considerations. First, IFN-γ can be produced by both donor dendritic cells following antigen recognition and donor T cells upon activation. In response to high concentrations of IFN-γ, MSC are induced to produce indoleamine 2,3-dioxygenase (IDO), the enzyme known to promote the immunosuppressive barrier at the maternal-fetal interface. Also, MSC treatment with IFN-γ in vitro has been observed to enhance MSC production of several immunosuppressive cytokines such as transforming growth factor-beta. MSC responded to the presence of this immunoregulatory protein by being stimulated to suppress GVHD.

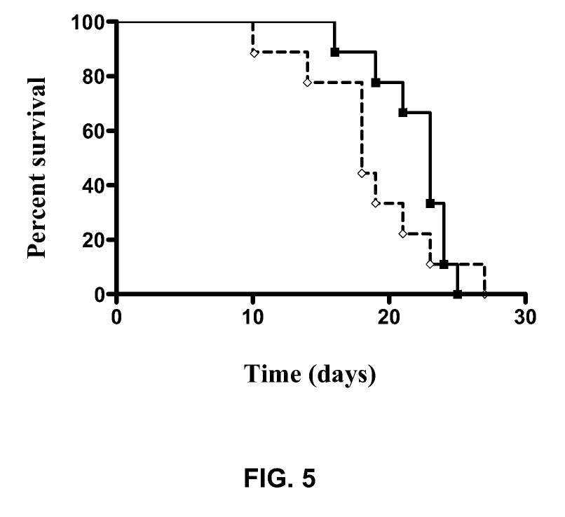

To determine whether IFN-γ played a role in MSC suppression of GVHD in vivo, donor splenocytes, incapable of producing IFN-γ, were infused to induce GVHD. In this system, the sources of IFN-γ were limited to the low numbers of antigen presenting cells within the bone marrow and host hematopoietic cells. GHVD-related mortality was rapid and severe; 100% mortality occurred prior to day 30 (FIG. 5). Addition of MSC had no effect. These data indicate that the absence of donor IFN-γ led to accelerated GVHD which could not be controlled by donor MSC.

### Example 8

### MSC Activation with IFN-γ

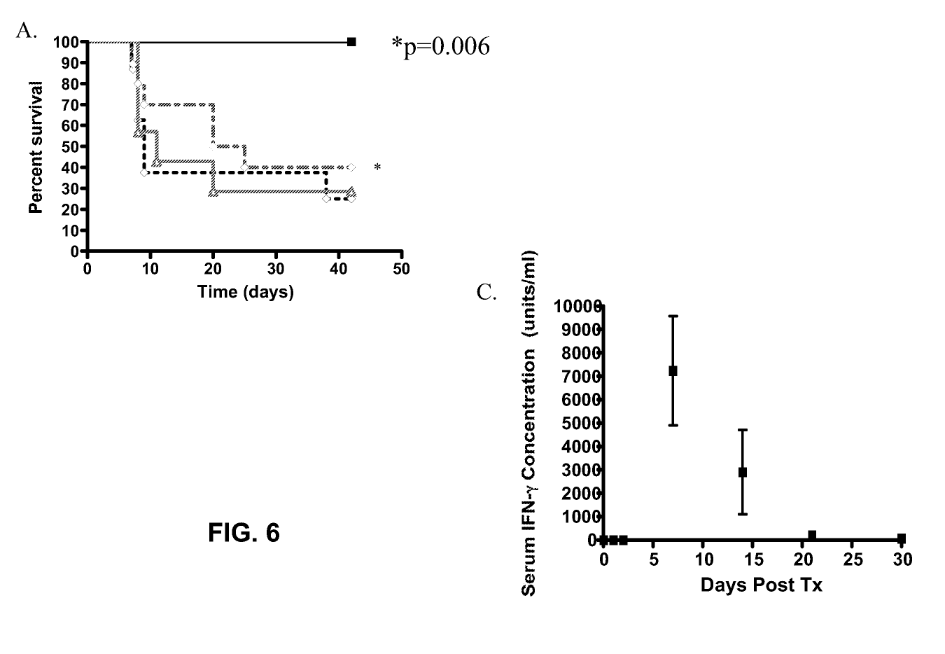

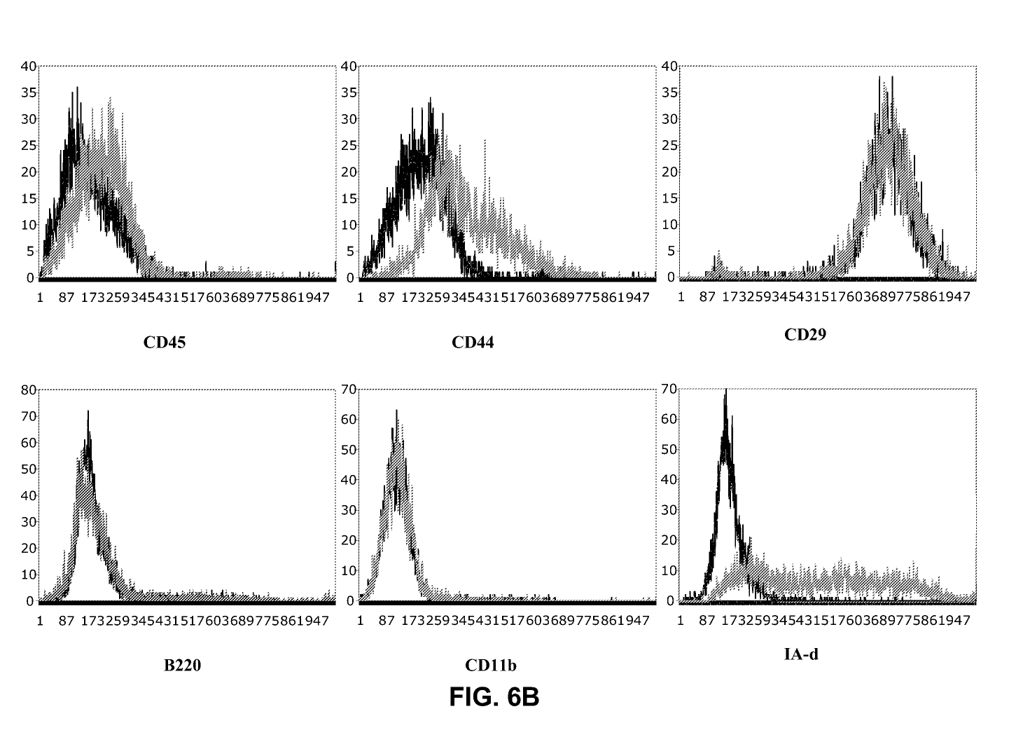

To further examine the ability of IFN-γ to initiate MSC suppression, MSC were treated with three concentrations of IFN-γ, 5, 50, or 500 units, prior to their administration on day 0. MSC might not be effective on day 0 because they failed to receive a sufficient IFN-γ stimulus. By pre-treating the MSC with IFN-γ, MSCs were activated for more efficient suppression of GVHD. When treated with either of the lower doses of IFN-γ, MSC were ineffective in significantly preventing GVHD mortality when compared to untreated MSC (FIG. 6A). In contrast, 500 units IFN-γ increased survival to 100%, (p=0.02) and this treatment was significantly better than MSC pre-treatment with 5 units (p=0.006) or 50 units (p=0.005). These data suggested that MSC suppression could be initiated with high dose but not low dose IFN-γ, thereby identifying a threshold of MSC activation in response to the immunoregulatory protein.

Suppression of T cells has also been observed by antigen presenting cells. To exclude the possibility that IFN-γ preferentially expanded an antigen presenting cell sub-population (found to be less than 3%) within the MSC preparation, a phenotypic analysis of the MSC following treatment with IFN-γ would show a distinct increase in the CD11b. B220 or CD45 positive population. MSC phenotype analyses were performed prior to each transplant experiment with IFN-γ-treated MSC (>10 times). No significant increases in CD45, CD11b, CD44, CD29 or B220 were noted, indicating there was no enrichment of an additional hematopoietic cell type to account for this effect (FIG. 6B). In response to IFN-γ treatment, MSC increased expression of MHC class II. These findings suggest MSC phenotype and function are modified in response to IFN-γ concentration.

### Example 9

### In Vivo Levels

With the identification of a critical level of IFN-γ required for MSC activation, the serum of transplant recipients were analyzed to determine when such levels were observed in vivo. Serum IFN-γ was measured by ELISA in pg on days 0, 1, 2, 7, 14, 21, 30 and was converted to units (conversion factor=10 units per pg, FIG. 6C). No serum IFN-γ was detected on day zero (at the time of transplant) or one. A small increase on day two, 2.7±2.7 units, was followed by a dramatic increase on day seven, 7232±2340 units. By day twenty-one, levels had decreased to 207±149 units, with 63±63 units noted on day 30. These data suggest, following transplantation, circulating IFN-γ at the time of transplant are not sufficient to activate MSC. By day 30 there is also insufficient circulating IFN-γ for MSC activation, providing a possible explanation for the lack of MSC efficacy when administered on day 30. To test whether IFN-γ alone had an effect on GVHD, we retreated 3 animals with 500 units IFN-γ iv on day 0 in the absence of MSC. All three animals survived to day 50, indicating a beneficial effect of this treatment on mortality, however all three developed severe GVHD with alopecia, weight loss, and scabbing, requiring euthanasia. These data suggest that while IFN-γ has a beneficial effect on GVHD mortality, activated MSC appear to have superior efficacy, since treatment with activated MSC on day 0 prevented the development of GVHD.

### Materials and Methods

Animals

Male BALB/c (H-2K^{d}) and C3H(H-2K^{k}) and female C57BL/6 (H-2K^{b}) mice were purchased from Fredericks NCI (Frederick, Md.) or Charles River (Wilmington, Mass.). Male C.129S7(B6)-Ifng^{tmITs}/J (INF-γ deficient) mice were purchased from Jackson Laboratories (Bar Harbor, Me.). All mice were housed in an AAALAC-accredited animal facility in microisolator cages equipped with autoclaved food and acidified water and were treated under conditions approved by the Animal Care Committee at the University of Illinois at Chicago (UIC).

Conditioning

Recipient mice were age matched (10-12 weeks) for each set of experiments and were exposed to lethal radiation 24 h prior to administration of donor BM and splenocytes. Irradiation was performed at the Department of Radiation Oncology at UIC after recipients were placed in a Lucite retainer for immobilization. The retainer was placed in a water equivalent phantom (box) 30×30×14.5 cc to ensure dose homogeneity during irradiation. Radiation was delivered via two portals (left and right) using a 6MV photon beam from a Clinac 2100EX (Varian Medical Systems, Palo Alto, Calif.) linear accelerator. A total of 1000 cGy at a dose rate of 100 cGy/min was delivered to the prescription point situated in the middle of the box. Across the box the delivered dose was homogeneous within +/−10%. The dose delivered to the animal was verified using ThermoLuminiscent Dosemeters (Harshaw/Bicron, Solon, Ohio) with agreement between measured and expected dose within +/−3.0%.

Bone Marrow, MSC, and Splenocyte Preparation

Following euthanasia, the BM contents of the femurs and tibia of donor Balb/C mice were flushed through a 40 μm filter (Becton Dickenson, Franklin Lakes, N.J.) into a 50 ml tube (Corning, Corning, N.Y.) containing MSC media, (40% alpha Modified Eagle Medium (αMEM, Invitrogen, Rockville, Md.), 40% F-12 nutrient mixture (Invitrogen), 10% fetal bovine serum (FBS, Valley Biomedical, Winchester, Va.), and 1% antibiotic-antimycotic solution (Invitrogen)). Splenocytes were flushed from spleens and filtered through a 40 μm filter (BD) into a 50 ml tube (Corning). BM cells and splenocytes were counted and resuspended in Hanks Balanced Salt Solution (HBSS, Invitrogen) to the appropriate dose and administered to recipient mice retro orbitally on Day 0 to irradiation in a total volume of 200 μl per recipient.

To obtain MSC, BM cells were plated at a density of 20×10⁶ per 9.6 cm² in MSC media at 37° C. in 5% CO₂ as previously described. The non-adherent population was removed after 72 h and the adherent cells were washed with fresh media and cultured for 7 additional days. The resulting adherent cells were harvested by incubating with 0.25% Trypsin (Invitrogen) for 6 min at 37° C. followed by gentle scraping. Cells were then incubated with biotinylated antibodies to mouse CD11b (10 μg per ml, e-biosciences, San Diego, Calif.) and CD45 (10 ug/ml, e-biosciences) for 30 min at 4° C. Positive cells were discarded after binding with MACS anti biotin beads and cohering to a magnetic column (Miltenyi Biotec, Auburn, Calif.). Negative cells were placed back into culture in Nunclon SoLo 185 cm² flasks (Nalge Nunc International, Rochester, N.Y.) at a density of 1×10⁶ cells per flask. A homogenous cell population was immediately following immunodepletion. The uniform phenotype was confirmed, based on the expression of CD29, CD44, and Seal, and the absence of hematopoietic (CD45, CD14, CD11b) markers. All antibodies were purchased from ebiosciences (San Diego, Calif.). The proportion of CD45+ cells in the MSC preparations used in the various experiments never exceeded 3% CD45+ cells. Prior to transplantation, cells had been passaged from 1 to 4 times. MSC primed with IFN-γ were plated at a density of 0.116×10⁶ per 9.6 cm² well in 6 well plates. This density is the equivalent to 1×10⁶ per 185 cm² flask. The 4 ml of MSC media in each well was supplemented with 500 units/ml recombinant murine IFN-γ (PeproTech, Rocky Hill, N.J.). On the day of transplantation (day 0), MSC were counted and resuspended at the appropriate dose in 100 μl HBSS per recipient in a 1 ml syringe (BD). MSC were injected retro-orbitally on either Day 2, 20 or 30 post irradiation. When co administered with BM and splenocytes on Day 0, the total volume remained 200 μl.

Flow Cytometry

MSC were characterized by Flow Cytometry (Cytomics FC 500, Beckman Coulter, Miami, Fla.). Briefly, MSC were resuspended at 1×10⁶ cells per ml in FACS buffer (PBS (Invitrogen) with 2% FBS (Valley Biomedical, Winchester, Va.) and 0.1% Sodium Azide (Sigma, St. Louis, Mo.)). Following Fc block (BD Pharmingen, San Jose, Calif.) at 1 μg/10⁶ cells for 15 min at 4° C. cells were stained with the following PE or FITC conjugated antibodies: H2 kd (SF1-1,1, BD), I-Ad (AMS-32.1, BD), CD2 (RM2-5, BD), CD3 (17A2, BD), CD4 (GK1.5, BD), CD8a (53-6.7, ebiosciences), CD11b (M1/70, ebiosciences) CD14 (rmC5-3, BD), CD44 (IM7, ebiosciences), CD45 (30-F11, BD), B220 (RA3-6B2, BD), Sca-1 (E13-161.7, BD), c-Kit (2B8, ebiosciences), Thy-1 (30-1112, BD), IFN-γ beta receptor (Abcam, Cambridge, Mass.) and appropriate isotype controls (ebiosciences or Abcam). Cells were also stained with a primary purified anti CD29 antibody (BD) at a concentration of 1 μg/10⁶ cells, washed with FACS buffer, and then stained with a secondary PE f(ab′)2 fragment donkey anti rat IgG (Jackson ImmunoResearch, West Grove, Pa.) at 0.5 μg/10⁶ cells. Flow analysis was performed following the acquisition of 10,000 events. MSC purity was verified within 2 days of transplantation. MSC were stained with FITC conjugated CD11b and CD45 as above.

GVHD Scoring

Mice were weighed twice weekly and monitored daily for survival and clinical evidence of GVHD (ruffled fur, cachexia, alopecia, and diarrhea). Control mice receiving no MSC and recipients of either 10⁵ or 5×10⁵ MSC administered on day 2 were euthanized on day 20 for histological examination. Liver, lung, colon, spleen, and skin were excised, sectioned, stained with hematoxylin and eosin, and examined and scored by two independent pathologists blinded to treatment groups. GVHD was scored on a scale from 0 (none) to 4.0 based on the scales reported by Ferrara (skin), Grass (liver, spleen), and Hill (colon). The scales for each tissue are defined as follows: for lung 0=normal, 0.5=minimal perivascular cuffing, 1.0=perivascular cuffing, 1 to 2 cells in thickness, involving up to 15% of vessels, 1.5=perivascular cuffing, 1 to 2 cells in thickness, involving up to 15% of vessels and infiltration into parenchyma proper, 2.0=perivascular cuffing, 2 to 3 cells in thickness, involving up to 15% of vessels and infiltration into parenchyma proper, 2.5=perivascular cuffing, 2 to 3 cells in thickness, involving 25% to 50% of vessels and infiltration into parenchyma proper, 3.0=perivascular cuffing, 4 to 5 cells in thickness, involving 25% to 50% of vessels, and infiltration into parenchyma proper, 3.5=perivascular cuffing, 6 to 7 cells in thickness, involving greater than 50% of vessels, peribronchiolar cuffing (4 to 5 cells), and infiltration into parenchyma proper with severe disruption of structure, 4.0=perivascular cuffing, 6 to 7 cells in thickness, involving greater than 50% of vessels, peribronchiolar cuffing (>6 cells), and infiltration into parenchyma proper with severe disruption of structure; for colon 0=normal, 0.5=occasional necrotic crypt cell, minimal infiltration in lamina propria and submucosa (colon), 1.0=necrotic cells in up to 15% of crypts, minor infiltration of up to 20% of lamina propria (1 to 2 cell thickness in intermucosal areas and submucosa, 1.5=necrotic cells in up to 15% of crypts, minor infiltration of less than or equal to one third of the lamina propria (1 to 2 cell thickness in intermucosal areas and submucosa), 2.0=necrotic cells in ≦25% of crypts, infiltration of less than or equal to one third of the lamina propria (3 cell thickness in intermucosal areas and submucosa), 2.5=necrotic cells in 25% to 50% of crypts, infiltration of less than or equal to one third of lamina propria (3 to 4 cell thickness in intermucosal areas and submucosa), 3.0=necrotic cells in greater than 50% of crypts, infiltration of lamina propria (5 to 6 cell thickness in intermucosal areas and submucosa) with loss of ≦25% of goblet cells, 3.5=necrotic cells in greater than 50% of crypts, infiltration of lamina propria resulting in displacement of ≦50% of mucosa with loss of 50% of goblet cells, 4.0=necrotic cells in greater than 50% of crypts, infiltration of lamina propria resulting in displacement of greater than 50% of mucosa with loss of 75% to 100% of goblet cells; for spleen 0=normal, 1.0=necrotic/apoptotic cells, up to 10 cells/mm2 of tissue, 1.5=necrotic/apoptotic cells, up to 10 cells/mm2 of tissue and occasional hemolysis, 2.0=necrotic/apoptotic cells, ≦20 cells/mm2 of tissue, and occasional hemolysis with abnormal architecture, 2.5=necrotic/apoptotic cells, ≦20 cells/mm2 of tissue, and hemolysis in ≦25% of the tissue with abnormal architecture, 3.0=necrotic/apoptotic cells, ≦40 cells/mm2 of tissue, hemolysis in 25% to 50% of tissue with abnormal architecture and areas of leukopenia involving ≦25% of tissue, formation of fibrous bands, 3.5=necrotic/apoptotic cells, up to 40 cells/mm2 of tissue, hemolysis evident in greater than 50% of tissue with abnormal architecture and areas of leukopenia involving 25% to 50% of tissue, formation of fibrous bands, 4.0=large areas of necrosis and hemolysis evident in greater than 50% of tissue with abnormal architecture and large areas of leukopenia involving greater than 50% of tissue; for skin 0=normal, 1.0=Basal keratinocyte ballooning, 2.0=sebaceous and adnexal infiltrate, 3.0=Loss of epidermis.

Quantification of Serum IFN-γ

Twenty B6 recipients underwent irradiation followed Balb/c bone marrow transplant with accessory splenocytes as described above. Three to five recipients underwent serum sampling for each timepoint, on days 0, 1, 2, 7, 14, 20, and 30. Sera was cryopreserved and batch analyzed using Quantikine Mouse IFN-γ Immunoassays by R&D Systems (Minneapolis, Minn., Cat#: MIFOO) as per manufacturer's instructions. A Multiskan

Ascent (Labsystems) plate reader provided OD read at 450 nm with 540 nm wavelength correction. Data were expressed as means with standard deviations.

Statistical Analyses

Treatment group sizes were designed based on an alpha of 0.05 and a power of 0.80. Each experiment was repeated at least 3 times with a minimum of n=10 per group, unless otherwise stated. Kaplan Meier curves (log rank test) were used to compare survival between treatment groups. ANOVA was used to compare GVHD scoring between groups. In all statistical analyses, a p value of 0.05 was deemed significant.

1. A method to prevent graft versus host disease in a mammal as result of a transplant, the method comprising:
(a) activating mammalian mesenchymal stem cells with IFN-γ; and
(b) administering the activated mammalian stem cells to the mammal who will host the graft.

2. The method of claim 1, wherein the activating is by a high dose of interferon gamma/ml.

3. The method of claim 2 wherein the high dose is greater than 50 units interferon gamma/ml.

4. The method of claim 3 wherein the high dose is about 2,000 or up to 20,000 for 2 hour units.

5. The method of claim 1, wherein the mesenchymal stem cells are from bone marrow.

6. The method of claim 5, wherein the bone marrow is human bone marrow.

7. The method of claim 2 wherein activating is achieved by at least 2 successive pulses of exposure to >50 units/ml IFN-γ.

8. The method of claim 3 wherein high doses are 500 units for 6 days.

9. The method of claim 1, wherein administering is approximately at the time of, or after, the transplant.

10. (canceled)

11. A method of initiating angiogenesis in a mammal comprising:
(a) obtaining a composition of human mesenchymal stem cells activated by an inflammatory cytokine; and
(b) administering the composition to a mammal need of tissue regeneration or organ repair.

12. A method to produce activated mesenchymal stem cells for use in tissue transplantation, the method comprising:
(a) obtaining mesenchymal stem cells; and
(b) activating the mesenchymal stem cells by culturing the cells with IFN-γ.

13. A composition of human mesenchymal stem cells activated by an inflammatory cytokine.

14. The composition of claim 13 wherein the inflammatory cytokine is IFN-γ.

15. (canceled)

16. The composition of claim 13, wherein the stem cells are activated with a dose greater than 50 units interferon gamma/ml.

17. The composition of claim 16, wherein stem cells are activated by at least 2 successive pulses of exposure to >50 units/ml IFN-γ within a 1-7 day period.

18. The composition of claim 13, wherein the mesenchymal stem cells are from bone marrow.

19. The composition of claim 18, wherein the bone marrow is human bone marrow.

20. The composition of claim 13 that prevents graft versus host disease in a mammal as result of a transplant.

21. A pharmaceutical composition comprising the composition of claim 13 that prevents graft versus host disease in a mammal as result of a transplant.
